(19) United States
(12) Patent Application Publication
(10) Pub. No.: US 2021/0219506 A1
(43) Pub. Date: Jul. 22, 2021

(54) SOLAR STEAM SYSTEMS FOR OPTIMIZED ALGAL BIOMASS PROCESSING

(71) Applicant: ExxonMobil Research and Engineering Company, Annandale, NJ (US)

(72) Inventors: Everett J O'Neal, Asbury, NJ (US); Anastasios Skoulidas, Pittstown, NJ (US); Patrick L Hanks, Bridgewater, NJ (US)

(21) Appl. No.: 17/082,195

(22) Filed: Oct. 28, 2020

Related U.S. Application Data
(60) Provisional application No. 62/963,273, filed on Jan. 20, 2020.

(51) Int. Cl. A01G 33/00; C12N 1/02; C12N 1/12

(52) U.S. Cl. CPC A01G 33/00; C12N 1/12; C12N 1/02

(57) ABSTRACT

Systems and methods for algae processing and, more particularly, to systems and methods for having integrated solar steam systems. Trapped heated air accumulated within the solar steam system, such as a greenhouse-enclosed solar steam system, is swept over a cultivated algae slurry in order to facilitate drying thereof and increasing the thermal efficiency of a biofuel algae facility.

### CROSS-REFERENCE TO RELATED APPLICATIONS

This application claims the benefit of priority from U.S. Provisional Application No. 62/963,273 filed Jan. 20, 2020, which is herein incorporated by reference in its entirety.

### FIELD OF THE INVENTION

The present disclosure is related to algal biomass processing and, more particularly, to systems and methods for integrating solar steam systems for optimized algal biomass processing.

### BACKGROUND OF THE INVENTION

Concerns about climate change, carbon dioxide (CO₂) emissions, and depleting mineral oil and gas resources have led to widespread interest in the production of biofuels from algae, including microalgae. As compared to other plant-based feedstocks, algae have higher CO₂ fixation efficiencies and growth rates, and growing algae can efficiently utilize wastewater, biomass residue, and industrial gases as nutrient sources.

Algae are photoautotrophic organisms that can survive, grow, and reproduce with energy derived entirely from the sun through the process of photosynthesis. Photosynthesis is essentially a carbon recycling process through which inorganic CO₂ combines with solar energy, other nutrients, and cellular biochemical processes to output gaseous oxygen and to synthesize carbohydrates and other compounds critical to the life of the algae.

To produce algal biomass, algae cells are generally grown in a water slurry comprising water and nutrients. The algae may be cultivated in indoor or outdoor environments, and in closed or open cultivation systems. Closed cultivation systems include photobioreactors, which utilize natural or artificial light to grow algae in an environment that is generally isolated from the external atmosphere. Such photobioreactors may be in a variety of shaped configurations, but are typically tubular or flat paneled. Open cultivation systems include natural and artificial ponds that utilize sunlight to facilitate photosynthesis. Artificial ponds are often shaped in circular or raceway-shaped (oval) configurations (referred to as “raceway ponds”).

Various processing methods exist for extracting lipids (oils) from harvested biomass for the production of fuel and other oil-based products. For example, one traditional method of extracting lipids includes processing an algal biomass into a paste, which may comprise between about 15-20% by weight (wt %) of algae. Thereafter, the paste is dried to a moisture level of about 10% or less, and the dried material further processed in an extruder or other mechanical shearing device to lyse the algae cells. Various chemicals (e.g., hexane) are used to extract the lipids from the lysed algae cells for use in biofuel production. As will be appreciated, the drying process alone requires an enormous amount of energy to reduce the moisture of the algal biomass to permit lipid extraction.

### SUMMARY OF THE INVENTION

The present disclosure is related to algal biomass processing and, more particularly, to systems and methods for integrating solar steam systems for optimized algal biomass processing.

In some aspects, a system is disclosed that includes a cultivation vessel containing an algae slurry therein for cultivation. A conduit is in fluid communication with the cultivation vessel to convey cultivated algae slurry from the cultivation vessel. A solar steam system generates trapped heated air that is swept over the cultivated algae slurry using an air current system in fluid communication with the conduit and the solar steam system.

In some aspects, a method is disclosed that includes the steps of cultivating an algae slurry in a cultivation vessel. Cultivated algae slurry is thereafter conveyed from the cultivation vessel using a conduit. Trapped heated air is generated within a solar steam system and swept over the cultivated algae slurry using an air current system in fluid communication with the conduit and the solar steam system.

### BRIEF DESCRIPTION OF THE DRAWINGS

The following figures are included to illustrate certain aspects of the present disclosure, and should not be viewed as exclusive examples. The subject matter disclosed is capable of considerable modifications, alterations, combinations, and equivalents in form and function, without departing from the scope of this disclosure.

FIG. 1 is a schematic diagram of an example a greenhouse-enclosed solar steam system, according to one or more aspects of the present disclosure.

FIG. 2 is a schematic diagram of an example of a greenhouse-enclosed solar steam system, according to one or more aspects of the present disclosure.

### DETAILED DESCRIPTION OF THE INVENTION

The present disclosure is related to algal biomass processing and, more particularly, to systems and methods for integrating solar steam systems for optimized algal biomass processing.

Biofuel production from harvested algal biomass derived from cultivated algae slurries offers sustainable energy solutions to reduce reliance on fossil fuels and reduce greenhouse gas emissions. To accomplish substantial economic, environmental, and societal impact, algae is typically cultivated in large-scale systems to produce large quantities of algal biomass. Such large-scale cultivation systems allow algae-derived biofuels to become more cost-effective and more widely available to the public. Currently, lipid extraction from algal biomass for biofuel processing is practiced by drying (referred to as “dewatering”) a harvested biomass to about 10% or less moisture, for example, before running the biomass through a shearing device (e.g., a steam expander, extruder with steam addition, and the like) to lyse the cells, at which point the biomass can undergo liquid-liquid extraction. The biomass drying process is often a major bottleneck in terms of production costs, energy costs, time, and environmental impact because of the substantial amount of drying required to achieve a desired moisture content.

Various aspects of the present disclosure describe systems and methods to reduce costs and energy consumption related to drying, or dewatering, algal biomass during processing for the production of biofuels. More specifically, various aspects of the present disclosure describe systems and methods that promote thermal efficiency by utilizing otherwise-wasted, hot swept air to aid in the drying process. One aspect of the present disclosure includes conveying an algae slurry through an environment for direct drying by swept air from a greenhouse-enclosed solar steam system.

As used herein, the term “algae slurry” or “algae water slurry,” and grammatical variants thereof, refers to a flowable mixture comprising at least water, algae cells, and algae nutrient media (e.g., phosphorous, nitrogen, and optionally additional elemental nutrients).

Algal sources for preparing the algae slurry include, but are not limited to, unicellular and multicellular algae. Examples of such algae can include, but are not limited to, a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In some examples, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurochrysis, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species. Any combination of the aforementioned algae sources may additionally be used to prepare an algae slurry.

The water for use in preparing the algae slurry may be from any water source including, but not limited to, fresh water, brackish water, seawater, wastewater (treated or untreated), synthetic seawater (e.g., water with added salts), and any combination thereof.

The algae nutrient media for use in forming an algae slurry may comprise at least nitrogen (e.g., in the form of ammonium nitrate or ammonium urea) and phosphorous. Other elemental micronutrients may also be included, such as potassium, iron, manganese, copper, zinc, molybdenum, vanadium, boron, chloride, cobalt, silicon, and the like, and any combination thereof.

As used herein, the term “cultivation vessel,” “vessel,” and grammatical variants thereof, refers to any of an open or closed algae cultivation system used for the growth of algal biomass, including bioreactors, photobioreactors, natural ponds, artificial ponds (e.g., raceway ponds), and the like, including combinations thereof.

As used herein, the term “greenhouse-enclosed solar steam system” or, simply, “solar steam system,” and grammatical variants thereof, refers to an enclosed greenhouse system capable of trapping heat and comprising one or more pipes having water running therethrough. Sunlight focuses on the water within the pipe, boils it, and generate steam therein. The solar steam systems described herein are integrated into an algae processing system and, swept air derived from the solar steam system is utilized to enhance thermal efficiency of biomass drying compared to traditional drying processes.

As used herein, the term “swept air,” and grammatical variants thereof, refers to air at an elevated temperature that is produced from and either collected or circulated within a greenhouse-enclosed solar steam system. For example, in some instances piped air from a photobioreactor is introduced into the greenhouse-enclosed solar steam systems described herein to simultaneously cool the photobioreactor (which may be prone to overheating) and introduce additional heated air into the greenhouse-enclosed solar steam system to be used as swept air for drying algae.

Solar steam systems are effective, low CO₂, commercially scaled sources of steam for use in various industrial processes. The solar steam systems described herein are greenhouse-enclosed to seal and protect the components of the system. For example, the greenhouse protects the system from the effects of wind, as well as from ash, sand, or dust contamination. These greenhouse-enclosed solar steam systems accumulated heat during operation, and this heated air is typically removed using large fans or other equipment for creating air current to maintain the greenhouse at reasonable temperatures to avoid damage to components of the system (e.g., plastics, electrical mechanisms, and the like). Accordingly, the heat value of the removed air is wasted. The present disclosure, however, integrates a greenhouse-enclosed solar steam system with an algal processing system to harness the heat value of the air accumulated within the greenhouse for use in facilitating drying of an algal biomass. This swept air from the greenhouse may be used for drying algal biomass alone or in combination with one or more other drying processes (e.g., steam drum drying).

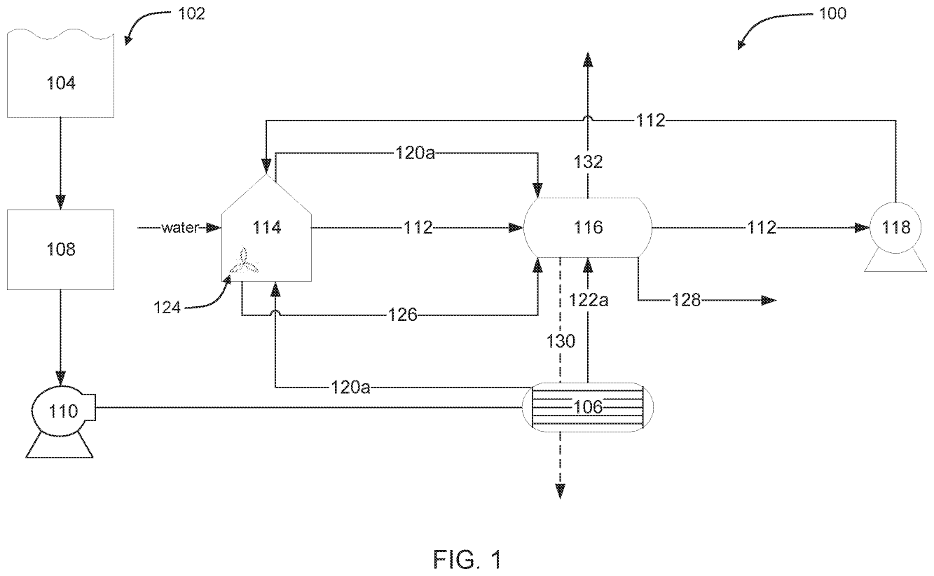

As described herein below, the swept air from the greenhouse may be directly flowed over harvested algal biomass to facilitate drying, which may encompass a pre-drying step or a final drying step. Referring now to FIG. 1, illustrated is a schematic diagram of an example system 100 for processing algae for use in biofuel production, according to one or more aspects of the present disclosure. As illustrated, the system 100 may include an algae cultivation vessel 102 (an open or closed cultivation vessel) configured to contain an algae slurry 104. The algae slurry 104 is maintained in conditions suitable for the growth within cultivation vessel 102.

The algae slurry 104 harvested from the cultivation vessel 102 may comprise about 1 to about 20 wt % algae (at least 10 wt % preferred) before being introduced into a heat exchanger 106. In some embodiments, to achieve a concentration of about 1 to about 30 wt % algae, the algae slurry 104 may pass through or may otherwise be processed in a separator 108 configured to increase the concentration of the algae within the algae slurry 104. The separator 108 may comprise, for example, a centrifuge or a membrane separator.

The system 100 may include a pump 110 configured to help convey the algae slurry 104 to the heat exchanger 106 from separator 108. Alternatively, the pump 110 may be omitted and hydrostatic pressure within the cultivation vessel 102 may be sufficient to convey the algae slurry 104 to the heat exchanger 106.

The heat exchanger 106 may comprise any type of heat exchanging device, apparatus, or system capable of increasing the temperature of the incoming algae slurry 104. In some aspects, the heat exchanger 106 may include, but is not limited to, a shell and tube heat exchanger, a plate heat exchanger, a plate and shell heat exchanger, an adiabatic wheel heat exchanger, a plate fin heat exchanger, a pillow plate heat exchanger, a fluid heat exchanger, a helical-coil heat exchanger, a spiral heat exchanger, or any combination thereof. In other aspects, the heat exchanger 106 may be powered, such as an electric or fired heat exchanger. In still other aspects, the heat exchanger 106 may employ renewable energy resources to heat the algae slurry 104, such as by use of a solar or geothermal heat exchanger, without departing from the scope of the disclosure.

The heat exchanger 106 may be configured to increase the temperature of the algae slurry 104 within a range of about 50° C. to about 200° C., and preferably about 70° C. to about 120° C., encompassing any value and subset therebetween. Moreover, the pump 110 and the heat exchanger 106 (or the heat exchanger 106 alone) may cooperatively increase the pressure of the algae slurry 104 within the heat exchanger 106. For example, in some embodiments, the pressure of the algae slurry 104 can be increased to a pressure ranging from about 0.1 bar to about 70 bar, or preferably about 0.1 bar to about 30 bar, encompassing any value and subset therebetween.

The high-pressure, high-temperature algae slurry 104 discharged from the heat exchanger 106 may then be dried by one or more methods, incorporating swept air from a greenhouse-enclosed solar steam system, as described in further detail hereinbelow.

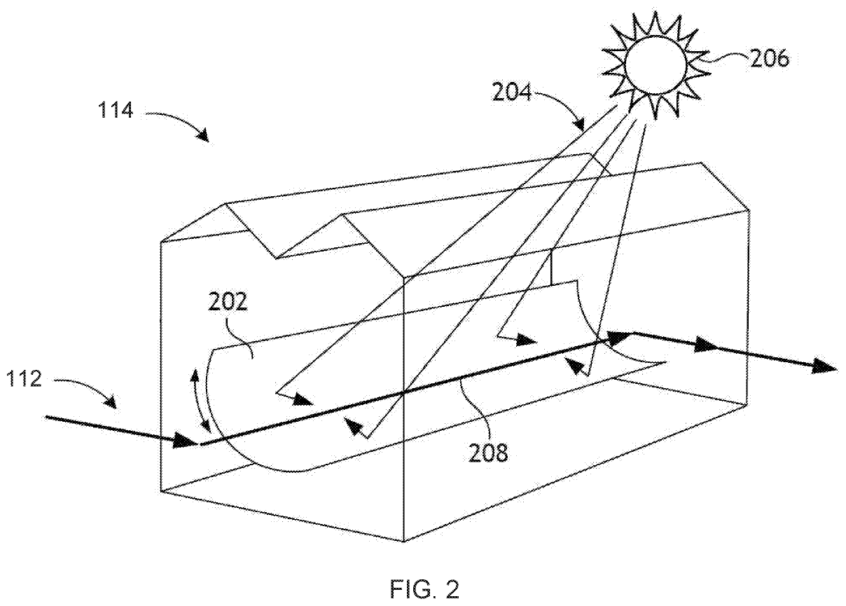

With continued reference to FIG. 1, the swept air is derived from greenhouse-enclosed solar steam system 114, described in more detail in FIG. 2 hereinbelow. Solar steam system 114 comprises a pipe that initially receives water (arrow shown in FIG. 1). The received water is boiled using solar rays within solar steam system 114 to create steam and, as a byproduct, heated air (which is used as swept air for further drying algal biomass according to the present disclosure). The steam is conveyed in steam conduit 112 (e.g., one or more pipes, hoses, channels, conveyer belts, troughs, and the like, with or without one or more pumps with suitable valving, if needed), exiting the solar steam system 114 for use, for example, in operation of one or more drum dryers 116 (one shown), or other equipment for processing algal biomass. The steam within steam conduit 112 may be thereafter conveyed to a boiler fed recirculation pump 118, where it may be condensed to liquid water by, for example, increasing pressure using pump 118 (e.g., with or without an incorporated compressor). Thereafter, the condensed steam is conveyed through steam conduit 112 back to the solar steam system 114 so that it may be again boiled to create steam (and heated air). As such, the initial water is recirculated without the requirement to continually add additional or new water to the solar steam system 114, and steam conduit 112 creates an enclosed conveyance line that prevents contamination of the water/steam therein (e.g., by algae, ash, sand, dirt, and the like).

As stated above, the high-pressure, high-temperature algae slurry 104 discharged from the heat exchanger 106 may be dried by one or more methods using swept air derived from the operation of solar steam system 114 (and recirculation loop comprising steam conduit 112 and associated equipment). More particularly, the algae slurry 104 may be pre-dried using swept air and/or finally dried using swept air.

For pre-drying, as shown in FIG. 1, the algae slurry 104 discharged from heat exchanger 106 may be conveyed in pre-drying conduit 120a to and through solar steam system 114. Pre-drying conduit 120a is configured to expose the algae slurry 104 to swept air within the solar steam system 114, for example, the pre-drying conduit 120a may be in association or itself be a conveyor belt or other conveyor system that exposes the algae slurry 104 to swept air within the solar steam system 114 for drying. Further, solar steam system 114 may be equipped with an air current system 124 for creating air current that drives the swept air therein over the algae slurry 104, as well as controlling humidity. The air current system 124 may include any number of fans or current generators, and may be located at any location relative to the solar steam system 114 to facilitate air movement. Alternatively, the evaporative process to remove moisture from the algae slurry 104 may cool the air within the solar steam system 114, creating a thermal driving force to move air therein, thus eliminating or reducing the need for air current system 124 (e.g., fewer fans or reduced fan speed). Such a system may result in density gradients by having an elevation difference between the solar steam generation system and the algae drying section within the greenhouse enclosure. Thereafter, the algae slurry 104 may exit the solar steam system 114 through pre-drying conduit 120a to drum dryer 116.

In an alternative or additional aspect, the algae slurry 104 may be finally dried using swept air. That is, in some aspects the algae slurry 104 may be both pre-dried and finally dried using swept air, or only one of pre-dried or finally dried using swept air, without departing from the scope of the present disclosure. As shown in FIG. 1, in one or more examples, the algae slurry 104 discharged from heat exchanger 106 may be conveyed in final drying conduit 122a to, for example, one or more drum dryers 116. The drum dryer 116 utilizes the steam in recirculation conduit 112 to create heat and the algae slurry 104 is flowed over the outside of a rotating drum, which dries the algae slurry 104 into a paste or flake material. Swept air may further be flowed across the algae slurry 104 as it is processed by the drum dryer 116 to increase drying effectiveness and efficiency (e.g., the drum dryer 116 could be used at a lower temperature because of the use of the swept air). The swept air may be conveyed from solar steam system 114 using one or more ducting conduits 126, in combination with air current system 124, to the drum dryer 116.

After the algae slurry 104 is dried using the drum dryer 116, with or without the use of swept air from the solar steam system 114, it may exit the drum dryer 116 or otherwise be removed therefrom, such as through conduit 128 as algal biomass. Alternatively, algal biomass may be collected directly from drum dryer 116 using a vessel or drum, for example without the need for conduit 128. In yet another alternative aspect, algal biomass may be sent back to heat exchanger 106 prior to collection to be used as a solid source of heat to increase the thermal efficiency of the heat exchanger 106. Water produced by the drum dryer 116 may further be removed therefrom, such as through conduit 132 and cooled to create fresh water and prevent any potential contamination within recirculation conduit 112.

In a specific aspect, for example, to operate an algal biomass facility to produce large amounts of biofuel, the system 100 would necessitate particular pressure and temperature requirements sufficient to flow over and dry algae slurry 104. If an algal biomass facility were designed to produce 10,000 barrels per day of biofuel, an approximate 140 megawatt solar steam system 114 would be required. Such a system could dramatically increase the thermal efficiency of an algal biomass simply by use of the typically wasted swept air, as described herein. For example, assuming a 50% thermal heat loss from heated air residing in the solar steam system 114 and a 50% thermal efficiency from drying algae using a fan-driven hot air system, a 25% reduction in solar steam requirement (energy) can be obtained according to the present disclosure, which could also decrease the footprint of the algal facility.

Referring now to FIG. 2, and with continued reference to FIG. 1, illustrated is a schematic diagram of an example of a greenhouse-enclosed solar steam system 114 (FIG. 1). In the illustrated example, the greenhouse-enclosed solar steam system 100 may comprise a solar heater that includes a parabolic mirror 202 configured and otherwise situated to reflect and redirect rays 204 emanating from the sun 206 toward a heat pipe 208. As water is conveyed through the heat pipe 208 in recirculation conduit 112 (FIG. 1), the parabolic mirror 202 concentrates (focuses) the reflected rays 204 onto the heat pipe 208 and thereby increases the temperature of the pipe 208 and, consequently, creates steam. While the heat pipe 208 is depicted as a straight section of pipe, it is contemplated herein that the heat pipe 208 include multiple interconnected turns and straight sections that allow the water to traverse a tortuous pathway as it heats to steam.

The particular configuration of the various components of system 100 in FIGS. 1 and 2 are non-limiting and thus not restricted to the configuration shown. Where not shown, each of the various conduits may be equipped with one or more valves or other flow control devices to block flow, allow flow, divert flow, or otherwise control flow of a flowable material within the conduits (e.g., water, algae slurry, algal biomass). Moreover, the particular path, length of path, and direction of path may be the same or different throughout the system 100 and is capable of various configurations provided that the swept air from solar steam system 114 can be utilized or otherwise collected to facilitate the drying of an algae slurry.

Accordingly, the system 100 of FIGS. 1 and 2 provides for methods of increasing the thermal efficiency of an algal facility by utilizing typically wasted swept air. Based on the system described herein, the methods include cultivating an algae water slurry within a cultivation vessel and contacting swept air derived from an integrated greenhouse-enclosed solar steam system with the algae slurry, the contacting being a pre-drying or final drying step.

### Examples Listing

The present disclosure provides, among others, the following examples, each of which may be considered as optionally including any alternate example.

Clause 1. A system comprising: a cultivation vessel to contain an algae slurry for cultivation; a conduit to convey the cultivated algae slurry from the cultivation vessel; a solar steam system to generate trapped heated air therewithin; and an air current system in fluid communication with the conduit and the solar steam system to sweep the trapped heated air over the cultivated algae slurry.

Clause 2. The system of Clause 1, wherein the conduit conveys the cultivated algae slurry through the solar steam system and the air current system sweeps the trapped heated air over the cultivated algae slurry within the solar steam system.

Clause 3. The system of Clause 1 or 2, wherein the conduit conveys the cultivated algae slurry over at least one drum dryer and the air current system sweeps the trapped heated air over the cultivated algae slurry as the cultivated algae slurry is conveyed over the at least one drum dryer.

Clause 4. The system of any of the preceding Clauses, wherein the solar steam system is a greenhouse-enclosed solar steam system.

Clause 5. The system of any of the preceding Clauses, wherein the solar steam system comprises a heat pipe that receives water, and a parabolic mirror situated to reflect and redirect rays emanating from the sun toward the heat pipe to heat the water and generate the trapped heated air.

Clause 6. The system of any of the preceding Clauses, further comprising a separator that fluidly interposes the cultivation vessel and the solar steam system to receive the cultivated algae slurry and reduce a concentration of water therein.

Clause 7. The system of Clause 6, wherein the separator is a centrifuge.

Clause 8. The system of any of the preceding Clauses, further comprising a heat exchanger that fluidly interposes the cultivation vessel and the solar steam system to heat and increase a pressure of the cultivated algae slurry prior to sweeping the trapped heated air over the cultivated algae slurry.

Clause 9. The system of any of the preceding Clauses, wherein the air current system comprises one or more fans.

Clause 10. The system of any of the preceding Clauses, wherein the cultivation vessel is a photobioreactor, and wherein heated air generated by the photobioreactor is introduced into the solar steam system, thereby forming a portion of the trapped heated air.

Clause 11. A method comprising: cultivating an algae slurry in a cultivation vessel; conveying the cultivated algae slurry from the cultivation vessel using a conduit; generating and trapping heated air within a solar steam system; and sweeping the trapped heated air over the cultivated algae slurry with an air current system in fluid communication with the conduit and the solar steam system.

Clause 12. The method of Clause 11, further comprising conveying, with the conduit, the cultivated algae slurry through the solar steam system and sweeping the trapped heated air, with the air current system, over the cultivated algae slurry within the solar steam system.

Clause 13. The method of Clause 11 or 12, further comprising conveying, with the conduit, the cultivated algae slurry over at least one drum dryer and sweeping the trapped heated air, with the air current system, over the cultivated algae slurry as the cultivated algae slurry is conveyed over the at least one drum dryer.

Clause 14. The method of any of Clauses 11 to 13, wherein the solar steam system is a greenhouse-enclosed solar steam system.

Clause 15. The method of any of Clauses 11 to 14, wherein the solar steam system comprises a heat pipe and a parabolic mirror, and further comprising reflecting and redirecting rays emanating from the sun toward the heat pipe to heat the water and generate the trapped heated air.

Clause 16. The method of any of Clauses 11 to 15, further comprising receiving the cultivated algae slurry and reducing a concentration of water therein using a separator that fluidly interposes the cultivation vessel and the solar steam system.

Clause 17. The method of Clause 16, wherein the separator is a centrifuge.

Clause 18. The method of any of Clauses 11 to 17, further comprising heating and increasing a pressure of the cultivated algae slurry using a heat exchanger that fluidly interposes the cultivation vessel and the solar steam system prior to sweeping the trapped heated air over the cultivated algae slurry.

Clause 19. The method of any of Clauses 11 to 18, wherein the air current system comprises one or more fans.

Clause 20. The method of any of Clauses 11 to 19, wherein the cultivation vessel is a photobioreactor, and further comprising introducing heated air generated by the photobioreactor into the solar steam system, thereby forming a portion of the trapped heated air.

Therefore, the disclosed systems and methods are well adapted to attain the ends and advantages mentioned as well as those that are inherent therein. The particular examples disclosed above are illustrative only, as the teachings of the present disclosure may be modified and practiced in different but equivalent manners apparent to those skilled in the art, having the benefit of the teachings herein. Furthermore, no limitations are intended to the details of construction or design herein shown, other than as described in the claims below. It is, therefore, evident that the particular illustrative examples disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A system comprising:
a cultivation vessel to contain an algae slurry for cultivation;
a conduit to convey the cultivated algae slurry from the cultivation vessel;
a solar steam system to generate trapped heated air therewithin; and
an air current system in fluid communication with the conduit and the solar steam system to sweep the trapped heated air over the cultivated algae slurry.

2. The system of claim 1, wherein the conduit conveys the cultivated algae slurry through the solar steam system and the air current system sweeps the trapped heated air over the cultivated algae slurry within the solar steam system.

3. The system of claim 1, wherein the conduit conveys the cultivated algae slurry over at least one drum dryer and the air current system sweeps the trapped heated air over the cultivated algae slurry as the cultivated algae slurry is conveyed over the at least one drum dryer.

4. The system of claim 1, wherein the solar steam system is a greenhouse-enclosed solar steam system.

5. The system of claim 1, wherein the solar steam system comprises a heat pipe that receives water, and a parabolic mirror situated to reflect and redirect rays emanating from the sun toward the heat pipe to heat the water and generate the trapped heated air.

6. The system of claim 1, further comprising a separator that fluidly interposes the cultivation vessel and the solar steam system to receive the cultivated algae slurry and reduce a concentration of water therein.

7. The system of claim 6, wherein the separator is a centrifuge.

8. The system of claim 1, further comprising a heat exchanger that fluidly interposes the cultivation vessel and the solar steam system to heat and increase a pressure of the cultivated algae slurry prior to sweeping the trapped heated air over the cultivated algae slurry.

9. The system of claim 1, wherein the air current system comprises one or more fans.

10. The system of claim 1, wherein the cultivation vessel is a photobioreactor, and wherein heated air generated by the photobioreactor is introduced into the solar steam system, thereby forming a portion of the trapped heated air.

11. A method comprising:
cultivating an algae slurry in a cultivation vessel;
conveying the cultivated algae slurry from the cultivation vessel using a conduit;
generating and trapping heated air within a solar steam system; and
sweeping the trapped heated air over the cultivated algae slurry with an air current system in fluid communication with the conduit and the solar steam system.

12. The method of claim 11, further comprising conveying, with the conduit, the cultivated algae slurry through the solar steam system and sweeping the trapped heated air, with the air current system, over the cultivated algae slurry within the solar steam system.

13. The method of claim 11, further comprising conveying, with the conduit, the cultivated algae slurry over at least one drum dryer and sweeping the trapped heated air, with the air current system, over the cultivated algae slurry as the cultivated algae slurry is conveyed over the at least one drum dryer.

14. The method of claim 11, wherein the solar steam system is a greenhouse-enclosed solar steam system.

15. The method of claim 11, wherein the solar steam system comprises a heat pipe and a parabolic mirror, and further comprising reflecting and redirecting rays emanating from the sun toward the heat pipe to heat the water and generate the trapped heated air.

16. The methods of claim 11, further comprising receiving the cultivated algae slurry and reducing a concentration of water therein using a separator that fluidly interposes the cultivation vessel and the solar steam system.

17. The method of claim 16, wherein the separator is a centrifuge.

18. The method of claim 11, further comprising heating and increasing a pressure of the cultivated algae slurry using a heat exchanger that fluidly interposes the cultivation vessel and the solar steam system prior to sweeping the trapped heated air over the cultivated algae slurry.

19. The method of claim 11, wherein the air current system comprises one or more fans.

20. The method of claim 11, wherein the cultivation vessel is a photobioreactor, and further comprising introducing heated air generated by the photobioreactor into the solar steam system, thereby forming a portion of the trapped heated air.
